(19) United States
(12) Plant Patent Application Publication
(10) Pub. No.: US 2003/0208809 P1
(43) Pub. Date: Nov. 6, 2003

(54) Prunus L. variety named 'Piku 3'

(75) Inventor: Brigitte Wolfram, Dresden (DE)

Correspondence Address: Russell D. Orkin, WEBB ZIESENHEIM LOGSDON ORKIN & HANSON, P.C., 700 Koppers Building, 436 Seventh Avenue, Pittsburgh, PA, 15219-1818, US

(73) Assignee: Bundesanstalt fur Zuchtungstorschung an Kulturpflanzen

(21) Appl. No.: 10/136,839

(22) Filed: May 1, 2002

(51) Int. Cl. A01H 5/00

(52) U.S. Cl. PLT/183

(57) ABSTRACT

A new and distinct Prunus rootstock compatible with other Prunus varieties and producing trees with reduced growth, adaptability to soil and high productivity.

### CLASSIFICATION

Prunus L.

### VARIETAL DENOMINATION

‘Piku 3’

### BACKGROUND OF THE INVENTION

The present invention comprises a new and distinct cultivar of bybrid Prunus known by the varietal name ‘Piku 3’. The new variety was discovered in 1972 in a selective breeding program designed to produce growth reducing rootstock for cherry trees in Dresden-Pilluitz, Germany as a cross between an unnamed, unpatented variety of Prunus pseudocerasus (female parent) and an unnamed, unpatented variety of Prunus incisa (male parent). The new variety differs from its parents in its growth habit. The following characteristic distinguish the new variety from Prunus avium varieties:

1. Three-way hybrid.

2. Reduces growth in grafted varieties by 20%.

3. Minimally precocious crop.

4. Adaptable to soil.

The new variety was first asexually reproduced via green cuttings in 1975 in Dresden-Pilluitz, Germany and subsequent propagation therein has confirmed that the characteristics of the new variety are stable and are transmitted to subsequent generations.

### DESCRIPTION OF THE DRAWINGS

The accompanying photographic drawings illustrate the new variety, with the color being as nearly true as is possible with color illustrations of this type.

FIG. 1 shows the tree of the new variety;

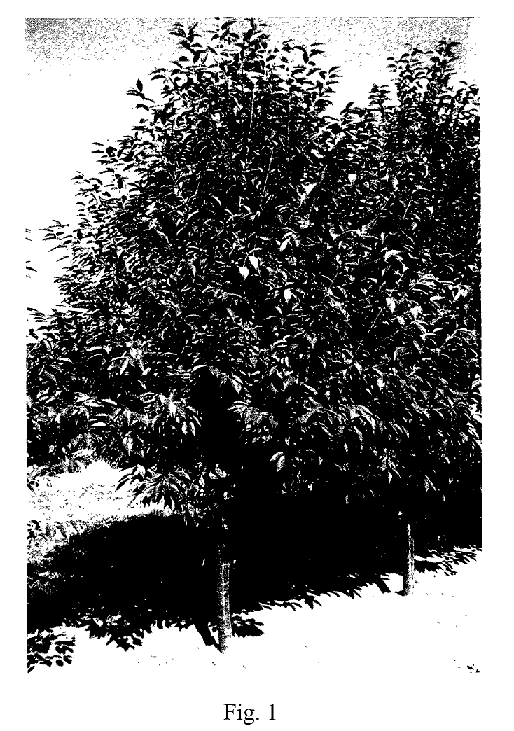

FIG. 2 shows a branch with leaves;

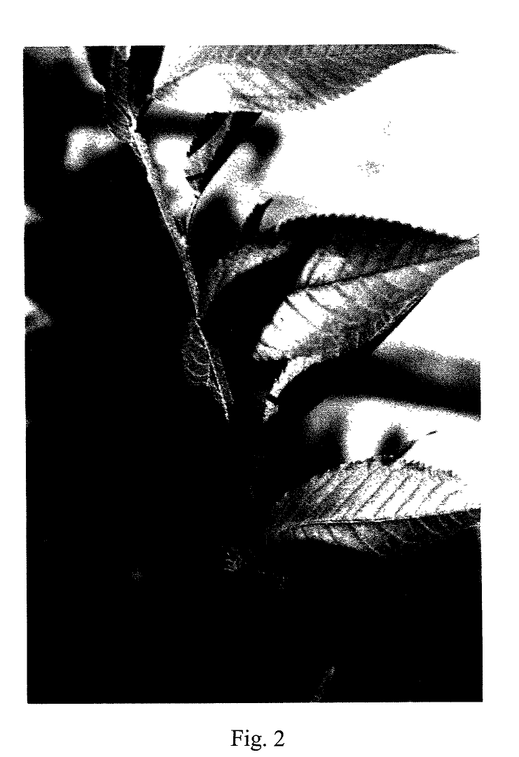

FIG. 3 shows a branch with flower buds; and

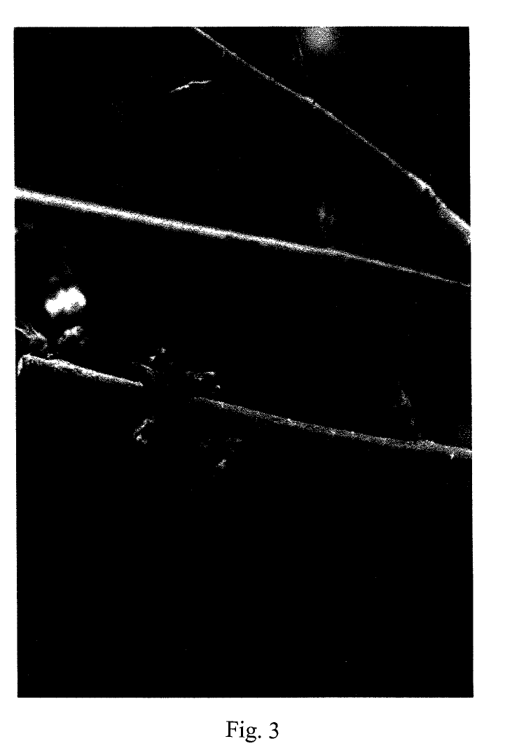

FIG. 4 shows a branch with opened flowers.

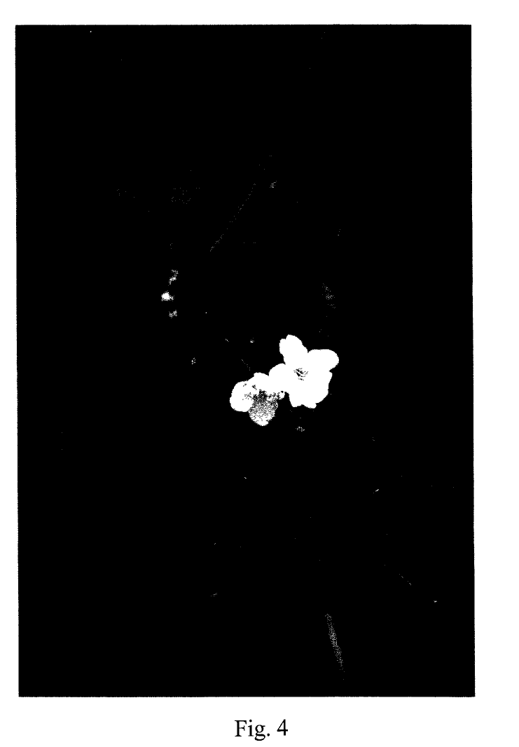

### DESCRIPTION OF THE PLANT

The following detailed description sets forth the characteristics of the new cultivar. The data which defines these characteristics were collected by asexual reproductions via green cuttings carried out in Dresden-Pilluitz, Germany. The color measurements were taken outdoors in Dresden-Pilluitz, Germany. Color designations are noted with reference to The Royal Horticultural Society Colour Chart. The new variety prefers dry soil and is resistant to PNRV and PDV. As a rootstock, it can be reproduced via softwood cuttings. The new variety anchors well with some root sprouts, good comparability with grafts and exhibits good growth vigor.

### TREE

Form: Upright, pryramidal.

Trunk:

Size.—Medium.

Bark color.—Brown.

Branches:

Surface texture.—Pubescent.

Bark color.—Brown.

New growth color.—Light-green red.

Mature growth color.—Dark.

Internode length.—About 2.3 cm.

Lenticels.—Many.

Bud.—Small.

Leaves:

Length.—8.7 cm.

Width.—4.0 cm.

Surface texture.—Pubescent.

Shape.—Elliptical.

Color.—Dark green.

Petioles:

Length.—1.2 cm.

Thickness.—Thin-medium.

Leaf glands:

Quantity.—1-2.

Position.—Under leaf end.

Size.—Small.

Color.—Green.

Stipules:

Quantity.—Rare.

Size.—Small.

Color.—Green.

Flowers:

Size.—Medium.

Color.—Light pink.

Number.—Many.

Sexual characteristics.—Diploid.

### FRUIT

Maturity date/location: Mid-season in Dresden, Pilluitz.

Size: Small; low crop yield.

Axial diameter: 1-2 cm.

Skin: Thin, soft, dark red

I claim:

1. A new and distinct variety of Prunus rootstock named ‘Piku 3’ substantially as shown and described.
